(19) United States
(12) Patent Application Publication
(10) Pub. No.: US 2023/0295555 A1
(43) Pub. Date: Sep. 21, 2023

(54) METHOD OF RECOVERING CELLS AND CELL RECOVERY APPARATUS

(71) Applicant: CANON KABUSHIKI KAISHA, Tokyo (JP)

(72) Inventors: FUMIO YAMAUCHI, Kanagawa (JP); KENGO KANAZAKI, Kanagawa (JP); SHINSUKE MOCHIZUKI, Kanagawa (JP); KEIICHIRO TSUBAKI, Kanagawa (JP)

(21) Appl. No.: 18/174,096

(22) Filed: Feb. 24, 2023

(30) Foreign Application Priority Data
Mar. 15, 2022 (JP) 2022-040723
Dec. 9, 2022 (JP) 2022-197317

(51) Int. Cl. C12M 1/00; C12M 1/42; C12M 1/12

(52) U.S. Cl. CPC C12M 47/02; C12M 35/04; C12M 37/02

(57) ABSTRACT

Provided are a method of recovering cells cultured on a substrate, the method including: a step 1 of adding a cell detachment solution containing a metal ion chelating agent to the substrate having the cells adhering thereto to bring the cells into contact with the cell detachment solution; a step 2 of applying ultrasonic vibration to the substrate and the cells to detach the cells from the substrate; and a step 3 of obtaining the cells singulated from the cells detached in the step 2, and a cell recovery apparatus capable of implementing the method of recovering cells.

### BACKGROUND OF THE INVENTION

### Field of the Invention

The present invention relates to a method of recovering cells and a cell recovery apparatus.

### Description of the Related Art

In recent years, a great paradigm shift has been occurring in drug development from conventional small molecule drugs and antibody drugs to cell drugs, which make use of cells themselves as drugs, and regenerative medicine, which is aimed at regeneration of tissues and organs. In the cell drugs and the regenerative medicine, large amounts of cells are needed, and in particular, there is a demand that adherent cells, which account for many biological tissues, be efficiently and stably supplied.

In culture of adherent cells, cells of interest are obtained through, for example, the steps of: culturing cells on a culture substrate such as a polystyrene dish; detaching the cells from the substrate; and recovering and washing the cells. When the cells are to be further proliferated, a so-called passaging operation is performed, in which part or all of the obtained cells are transferred to a fresh substrate and cultured again. Of the series of steps, the detachment step of detaching the cells from the substrate poses a significant barrier to efficient and stable supply of the cells.

In general, a method involving using a protease such as trypsin is widely used for detachment of cells from a substrate. In this method, the protease is allowed to act to degrade a protein involved in binding between the cells and the substrate, and then the cells are completely detached by an operation, such as tapping or pipetting. However, this method poses a concern that many other proteins present on surfaces of the cells may be concurrently degraded by the protease to reduce the quality of the cells.

Meanwhile, there is known a method of mechanically detaching cells from a culture vessel without using any protease.

In Yuta Kurashina et al., Communications Biology, 2, 393 (2019), there is a proposal of a cell detachment method involving using an ultrasonic wave. In Yuta Kurashina et al., Communications Biology, 2, 393 (2019), there is a disclosure of a method of detaching cells from a substrate through use of a cell detachment device including an ultrasonic transducer formed of a ring-shaped piezoelectric element. According to the method described in Yuta Kurashina et al., Communications Biology, 2, 393 (2019), the cells can be detached from the substrate without using any protease, and hence high-quality cells can be provided.

In Japanese Patent No. 4775218, there is a proposal of a mechanical cell detachment method that avoids use of trypsin.

In addition, in Japanese Patent No. 4775218, with a view to turning a cell mass detached from a substrate into singulated cells, there is a disclosure of a method involving passing a cell suspension containing the cell mass through a small tube (inner diameter: 0.2 mm or more and 1.0 mm or less).

In the cell detachment method described in Yuta Kurashina et al., Communications Biology, 2, 393 (2019), the cells detached from the substrate were brought into a state in which the cells were associated with each other, resulting in a cell mass in some cases. The state of being a cell mass poses problems in that efficiency of subsequent growth is reduced, and that cell counting becomes difficult.

In the method described in Japanese Patent No. 4775218, when the cell mass detached from the substrate was large, or when an aggregate of cell masses was present, the small tube was clogged in some cases. In addition, the cells were not sufficiently singulated in some cases, and the singulated cells had a low survival rate in some cases.

Accordingly, there have been demands for a more effective method of singulating cells and a simple apparatus capable of recovering singulated cells, for cells mechanically detached from a substrate without use of any protease.

### SUMMARY OF THE INVENTION

An object of the present invention is to provide a method of recovering cells and a cell recovery apparatus each of which solves the problems in the related art described above.

That is, the object is to provide a method of recovering cells and a cell recovery apparatus each of which is capable of turning a cell mass detached from a substrate into singulated cells effectively and with a high survival rate in a method involving detaching cells from the substrate through use of an ultrasonic wave and recovering the singulated cells.

A method of recovering cells according to one aspect of the present invention is a method of recovering cells cultured on a substrate, the method including: a step 1 of adding a cell detachment solution containing a metal ion chelating agent to the substrate having the cells adhering thereto to bring the cells into contact with the cell detachment solution; a step 2 of applying ultrasonic vibration to the substrate and the cells to detach the cells from the substrate; and a step 3 of obtaining the cells singulated from the cells detached in the step 2.

In addition, a cell recovery apparatus according to another aspect of the present invention is a cell recovery apparatus configured to recover cells cultured on a substrate, the cell recovery apparatus including: a medium exchange section configured to add a cell detachment solution containing a metal ion chelating agent to the substrate having the cells adhering thereto to bring the cells into contact with the cell detachment solution; an ultrasonic irradiation section configured to apply ultrasonic vibration to the substrate and the cells; and a singulated cell obtaining section configured to obtain the cells singulated from the cells detached from the substrate.

Further features of the present invention will become apparent from the following description of exemplary embodiments with reference to the attached drawings.

### BRIEF DESCRIPTION OF THE DRAWINGS

FIG. 1 is a flowchart for illustrating an example of the flow of a method of recovering cells according to an embodiment of the present invention.

FIG. 2 is a conceptual view of a cell recovery apparatus according to an embodiment of the present invention.

### DESCRIPTION OF THE EMBODIMENTS

The inventors have made extensive investigations on a technique capable of simply turning a cell mass detached from a substrate into singulated cells in a method of detaching and recovering cells from the substrate through use of an ultrasonic wave without use of any protease. As a result, the inventors have found that the following method can dissociate cells from a cell mass while maintaining a high survival rate, to thereby effectively provide singulated cells.

That is, the method is a method involving using a cell detachment solution containing a metal ion chelating agent as a cell detachment solution to be used in the detachment of cells from a substrate and further using an ultrasonic wave to detach the cells from the substrate, and then obtaining cells singulated from a cell mass detached from the substrate.

Herein, as a method of obtaining singulated cells, there is given a method involving passing a cell suspension containing the cell mass detached from the substrate through a mesh filter. Hitherto, the mesh filter has been used for the purpose of isolating singulated cells by removing large debris and cell masses remaining without being dissociated at the time of enzymatic treatment of cells or a tissue. In an embodiment of the present invention, the mesh filter is used for applying a shear force (detaching force) to the cell mass by passing a cell suspension, which is obtained through incorporation of the cells detached from the substrate into the cell detachment solution containing a metal ion chelating agent, through the mesh filter. When the mesh filter is used in the embodiment of the present invention as described above, there can be provided a novel cell recovery method capable of providing singulated cells without reducing the survival rate of the cells.

Next, the inventors have investigated a technique for further reducing damage to cells caused by passage through the mesh filter. Cells can be easily singulated by a related-art method involving using a protease, and hence no effective technique has heretofore been found for protecting cells from the shear force in the step of obtaining singulated cells after the cells have been detached from the substrate by a physical technique. The inventors have made extensive investigations, and as a result, have found that the addition of serum to the cell detachment solution (cell suspension) containing the cells detached through use of an ultrasonic wave can allow the cells to have resistance to the shear force in the subsequent step. Thus, the inventors have succeeded in turning the cell mass into singulated cells with a higher survival rate.

The embodiment of the present invention is described in more detail below.

(Action and Effect)

The method of recovering cells according to the embodiment of the present invention includes a mechanical detachment method based on ultrasonic vibration. It is important for the method of recovering cells according to the embodiment of the present invention to include a process involving detaching cells adhering to the substrate without causing damage thereto, and singulating the cells.

The cell detachment solution contains a metal ion chelating agent, and hence can weaken adhesion between the cells in an adhering state. Consequently, when the cells are detached through ultrasonic vibration, the cells can be detached through small vibration, and hence damage to the cells can be suppressed. Further, the metal ion chelating agent can contribute to the cell singulation in the subsequent step. That is, the weakening of the adhesion between the cells can reduce the shear force on the cells that is generated when the cell mass is turned into singulated cells.

As described later, it is particularly preferred that the cell mass be turned into singulated cells after the addition of serum to the cell suspension. This is conceivably because a protein contained in the serum has a function of protecting cells. Presumably as a result of the combination of a chemical separation action exhibited by the metal ion chelating agent, a physical separation action of applying a shear force to the cell mass, and a biological protection action exhibited by the serum, the cell mass obtained through mechanical detachment has been able to be turned into singulated cells in a particularly effective manner.

The method of recovering cells according to the embodiment of the present invention is a method of recovering cells cultured on a substrate, and includes the following steps 1 to 3.

The step 1 is a step of adding a cell detachment solution containing a metal ion chelating agent to the substrate having the cells adhering thereto to bring the cells into contact with the cell detachment solution.

The step 2 is a step of applying ultrasonic vibration to the substrate and the cells to detach the cells from the substrate.

The step 3 is a step of obtaining the cells singulated from the cells detached in the step 2.

(Culture Conditions for Cells)

Culture conditions for the cells may be appropriately selected in accordance with the cells to be cultured. In general, an appropriate medium is added into a dish serving as the substrate, and the cells are seeded therein at from about 1.0×10¹ cells/cm² to about 5.0×10⁴ cells/cm² and are cultured under an environment at 37° C. and a CO2 concentration of 5%. At this time, the culture is preferably performed until a cell coverage area ratio in the substrate of from about 70% to about 80%, i.e., a so-called subconfluent state is achieved.

The steps 1 to 3 are described in detail below.

(Step 1)

The step 1 in the method of recovering cells according to the embodiment of the present invention is a step of adding a cell detachment solution containing a metal ion chelating agent to the substrate having the cells adhering thereto to bring the cells into contact with the cell detachment solution.

The contact of the cells with the cell detachment solution reduces intercellular binding and an adhesion force between the cells and the substrate. The step 1 enables the cells to be detached from the substrate with high efficiency through the ultrasonic vibration applied in the next step 2. In other words, when the step 1 is not performed, the cells cannot be detached with high efficiency.

A period of time for which the cells are brought into contact with the cell detachment solution in the step 1 is not particularly limited, but is, for example, from 1 second to 24 hours, preferably from 10 seconds to 12 hours, more preferably from 30 seconds to 6 hours from the viewpoints of effectively detaching the cells and increasing the survival rate of the cells.

The temperature of the cell detachment solution to be used in the step 1 is not particularly limited, but is preferably 10° C. or more and 40° C. or less from the viewpoint of maintaining the cell survival rate, and is particularly preferably 30° C. or more and 40° C. or less, which is the optimal temperature for cell growth.

Before and after the step 1, the cells may be washed with a buffer. For example, when the cultured cells are to be detached from the substrate, the effect of the cell detachment solution may be increased by first removing the growth medium and then washing the cells in a state of adhering to the substrate with a buffer before adding the cell detachment solution. This is because the medium for growth generally contains a factor that inhibits cell detachment, such as serum.

(Cells)

The cells in the embodiment of the present invention are not particularly limited as long as the cells can adhere to, and be cultured on, a culture substrate in vitro. Examples of the cells include: various cultured cell lines, such as Chinese hamster ovary-derived CHO cells, mouse connective tissue L929 cells, mouse skeletal muscle myoblasts (C2C12 cells), human fetal lung-derived normal diploid fibroblasts (TIG-3 cells), human embryonic kidney-derived cells (HEK293 cells), human alveolar basal epithelial adenocarcinoma-derived A549 cells, and human cervical cancer-derived HeLa cells; epithelial cells and endothelial cells making up various tissues and organs in living bodies, skeletal muscle cells, smooth muscle cells, and cardiac muscle cells exhibiting contractility, neuronal cells and glial cells making up the nervous system, fibroblasts, and hepatic parenchymal cells, hepatic nonparenchymal cells, and adipocytes involved in metabolisms in living bodies; as cells having differentiation potency, various stem cells, such as induced pluripotent stem (iPS) cells, embryonic stem (ES) cells, embryonic germ (EG) cells, embryonal carcinoma (EC) cells, mesenchymal stem cells, hepatic stem cells, pancreatic stem cells, skin stem cells, muscle stem cells, and germline stem cells, and progenitor cells of various tissues; and cells obtained by inducing differentiation of cells having differentiation potency. The method of recovering cells according to the embodiment of the present invention is suitable for cells having strong intercellular binding, cells having a high adhesion force to the substrate, and cells having high trypsin sensitivity out of those cells. The method is particularly suitable for cells that are liable to form a cell mass, for example, mesenchymal stem cells.

(Substrate)

The substrate in the embodiment of the present invention means a culture vessel to be used for cell culture. The culture vessel is not particularly limited as long as the culture vessel is a cell-adhesive culture vessel, and examples thereof include a flask, a flask for tissue culture, a dish, a Petri dish, a dish for tissue culture, a multidish, a microplate, a multiwell plate, a multiplate, a Petri plate, a culture bag, and a bottle.

A material for the substrate in the embodiment of the present invention only needs to be a material that is chemically stable and capable of culturing desired cells. Examples of the material for the substrate include polyethylene, polypropylene, polycarbonate, polystyrene, polyvinyl chloride, nylon, polyurethane, polyurea, polylactic acid, polyglycolic acid, polyvinyl alcohol, polyvinyl acetate, poly(meth)acrylic acid, poly(meth)acrylic acid derivatives, polyacrylonitrile, poly(meth)acrylamide, poly(meth)acrylamide derivatives, polysulfone, cellulose, cellulose derivatives, polysilicone, polymethylpentene, glass, and metals.

Of those, polystyrene is preferred as the material for the substrate. The expression “(meth)acrylic” as used herein means “acrylic and methacrylic.” For example, “poly(meth)acrylic acid” means “polyacrylic acid and polymethacrylic acid.”

(Cell Detachment Solution Containing Metal Ion Chelating Agent)

The cell detachment solution to be used in the embodiment of the present invention contains a metal ion chelating agent (hereinafter sometimes referred to as “chelating agent”). Through use of the cell detachment solution containing the chelating agent, the cells can be effectively detached from the substrate through the ultrasonic vibration in the step 2, and treatment for obtaining singulated cells in the step 3 to be described later can also be effectively performed. When the cell detachment solution does not contain the chelating agent, the detachment ratio of the cells is reduced, and besides, the treatment for obtaining singulated cells cannot be sufficiently performed.

(Chelating Agent)

The chelating agent in the embodiment of the present invention is not particularly limited, but examples thereof may include ethylenediaminetetraacetic acid, ethylenediamine, ethylenediaminetetramethylenephosphonic acid, glycol ether diaminetetraacetic acid, nitrilotriacetic acid, diethylenetriaminepentaacetic acid, iminodiacetic acid, dihydroxyethylglycine, dicarboxymethylglutamic acid, ethylenediaminedisuccinic acid, etidronic acid, citric acid, gluconic acid, and phosphonobutanetriacetic acid. The chelating agent is preferably a chelating agent that forms a chelate with a divalent cation, particularly preferably a chelating agent that forms a chelate with each of Ca²⁺ and Mg²⁺, most preferably ethylenediaminetetraacetic acid out of those chelating agents. When ethylenediaminetetraacetic acid is used as the chelating agent, the pH of the cell detachment solution is preferably 7.0 or more and 8.0 or less. This is because, when the pH is higher within a neutral region, in which the survival rate of the cells can be kept high, the chelating ability of ethylenediaminetetraacetic acid can be enhanced, and hence the detachment ratio can be further increased. The chelating agents may be used alone or in combination thereof.

The content of the chelating agent in the cell detachment solution is preferably 0.01 mM or more and 5.0 mM or less. When the content of the chelating agent in the cell detachment solution falls within this range, a chelating effect can be reliably obtained, and a reduction in activity of the cells due to the presence of an excess of the chelating agent can be suppressed.

(Cell Detachment Solution)

The cell detachment solution to be used in the embodiment of the present invention is a solution containing a metal ion chelating agent, and is a solution to be used for detaching the cells from the substrate. The solution containing the metal ion chelating agent is not limited as long as the solution is an aqueous solution that does not impair the activity of the cells, and may contain various salts and factors, and a medium. In addition, the cell detachment solution may contain serum or an antibiotic. For example, the cell detachment solution may contain, as the above-mentioned factors, which are not particularly limited, for example, a protein, such as albumin, insulin, or transferrin, selenium, an antibiotic, a sugar such as glucose, a lipid, and a synthetic polymer.

The pH of the cell detachment solution is preferably in the neutral region. This is because the neutral region is suited for stably keeping the cells, and allows the survival rate of the cells to be stably kept high. The pH of the cell detachment solution may be appropriately adjusted with hydrochloric acid, sodium hydroxide, or the like. In addition, various buffers are suitably used for stably keeping the pH of the cell detachment solution.

The viscosity of the cell detachment solution is preferably 1.80 mPa·s or less. This is because a flow of the cell detachment solution to be generated by the ultrasonic vibration is not inhibited, and hence the detachment ratio can be kept high. The viscosity of the cell detachment solution may be appropriately adjusted by, for example, adding a polymer or a sugar.

With regard to the amount of the protease in the cell detachment solution, the content of the protease with respect to the total mass of the cell detachment solution is preferably 0.0005 mass % or less. In addition, it is more preferred that the cell detachment solution be substantially free of any protease. This is because, as described in the “Description of the Related Art” section, the protease degrades part of the cells, and hence the incorporation of the protease into the cell detachment solution can increase the detachment ratio but may reduce the quality of the cells.

Herein, the “protease” is, for example, one that degrades part of the cells to facilitate the detachment of the cells from the substrate. Examples of the protease include trypsin, accutase, collagenase, natural protease, chymotrypsin, elastase, papain, pronase, and recombinants thereof

(Buffer)

In the embodiment of the present invention, any buffer may be used for the cell detachment solution without any limitation as long as the neutral region can be kept. Examples of the buffer include a Tris buffer such as a Tris-HCl buffer, a phosphate buffer, a HEPES buffer, a citrate-phosphate buffer, a glycylglycine-sodium hydroxide buffer, a Britton-Robinson buffer, and a GTA buffer. Of those, a phosphate buffer, which is close to an in vivo environment, is preferred, and phosphate buffered saline (PBS) obtained by adjusting the phosphate buffer so as to be isotonic to an intracellular fluid is more suitably used.

(Medium)

The kind of the medium that the cell detachment solution may contain is not particularly limited, and examples thereof include Dulbecco's Modified Eagles's Medium (DMEM), Ham's Nutrient Mixture F12, DMEM/F12 medium, McCoy's 5A medium, Eagles's Minimum Essential Medium (EMEM), alpha Modified Eagles's Minimum Essential Medium (aMEM), Minimum Essential Medium, RPMI 1640 medium, Iscove's Modified Dulbecco's Medium (IMDM), MCDB 131 medium, Williams' Medium E, IPL 41 medium, Fischer's medium, StemSpan H3000 (manufactured by STEMCELL Technologies), StemSpan SFEM (manufactured by STEMCELL Technologies), Stemline II (manufactured by Sigma-Aldrich), Endothelial Cell Growth Medium 2 Kit (manufactured by PromoCell), Mesenchymal Stem Cell Growth Medium 2 (manufactured by PromoCell), MSCGM Bullet Kit (manufactured by Lonza), mTeSR1 or mTeSR2 medium (manufactured by STEMCELL Technologies), REPRO FF or REPRO FF2 (manufactured by REPROCELL), NutriStem medium (manufactured by Biological Industries), and MF-Medium mesenchymal stem cell growth medium (manufactured by TOYOBO Co., Ltd.).

Of those, a medium suited for the culture of each type of cells is preferably used for the cell detachment solution.

(Serum)

Examples of the serum that the cell detachment solution may contain include fetal bovine serum (FBS), calf serum, adult bovine serum, horse serum, sheep serum, goat serum, porcine serum, chicken serum, rabbit serum, and human serum. Of those, FBS is conveniently used because of its ease of availability. In addition, the cell detachment solution may contain a serum-free medium free of any untreated or unpurified serum and containing a purified blood-derived component or animal tissue-derived component (e.g., a growth factor).

(Antibiotic)

Examples of the antibiotic that the cell detachment solution may contain include penicillin, streptomycin, ampicillin, carbenicillin, tetracycline, bleomycin, actinomycin, kanamycin, actinomycin D, and amphotericin B.

(Step 2)

The step 2 is a step of applying ultrasonic vibration to the cells to detach the cells from the substrate. The step 2 enables the cells adhering to the substrate to be effectively detached. In other words, when the step 2 is not performed, the cells cannot be detached with a high detachment ratio.

The step 2 may include a step of applying an external stimulus such as a convective flow in the cell detachment solution as well as the above-mentioned step of applying ultrasonic vibration. As a method of generating a convective flow in the cell detachment solution, there are given, for example, pipetting and methods involving using a pump and a stirring blade.

A period of time for which the ultrasonic vibration is applied in the step 2 is not particularly limited, but is, for example, from 1 second to 1 hour, preferably from 5 seconds to 30 minutes, more preferably from 10 seconds to 15 minutes from the viewpoints of effectively detaching the cells and increasing the survival rate of the cells.

An environmental temperature in the step 2 is not particularly limited, but is preferably 20° C. or more and 40° C. or less, more preferably 30.0° C. or more and 37.5° C. or less from the viewpoint of maintaining the cell survival rate.

That is because, when the environmental temperature in the step 2 is set to from 20° C. to 40° C., the temperature at the time of cell detachment becomes close to the temperature at the time of culture, and hence the influence of temperature change on the cells can be reduced, with the result that the survival rate of the cells can be kept high.

(Ultrasonic Vibration)

In the embodiment of the present invention, the “ultrasonic vibration” is vibration having a frequency of 15 kHz or more. Any vibrating technique may be used without any particular limitation as long as the technique can apply ultrasonic vibration to the substrate and the cells. As an example, the ultrasonic vibration may be applied to the substrate and the cells by bringing an ultrasonic transducer including a piezoelectric element such as lead zirconate titanate (PZT) into contact with the substrate having the cells cultured thereon, and inputting, for example, an arbitrary frequency and voltage into an ultrasonic vibration member.

In this case, as the ultrasonic transducer, there is suitably used, for example, a Langevin-type transducer, or a transducer of a flat plate shape obtained by bonding a piezoelectric element to a glass plate, a metal plate, or the like with an adhesive or the like. A liquid such as water or a gel-like substance such as glycerol may be interposed between the ultrasonic transducer and the substrate. The formation of a layer of such liquid or gel-like substance can prevent an air layer from being formed between the ultrasonic transducer and the substrate.

(Step 3)

The step 3 is a step of obtaining the cells singulated from the cells detached from the substrate.

Treatment for obtaining singulated cells (hereinafter sometimes referred to as “cell singulation treatment”) is not particularly limited as long as the treatment does not include dissociation of cells through enzymatic treatment, but is preferably performed, for example, by a method involving passing a cell suspension containing a cell mass through a mesh filter. In addition, the cell singulation treatment is more preferably performed, for example, with a cell suspension containing serum, and for example, a method involving passing a cell suspension containing serum (also containing a metal ion chelating agent) through a mesh filter is a particularly preferred mode. In addition, the cell singulation treatment may be performed by adjusting the concentration of the metal ion chelating agent in a cell suspension containing the cells detached in the above-mentioned step 2 to 1.0 mM or more, and then applying a shear force to the cells. For example, the cell singulation treatment may be performed by adding a metal ion chelating agent at a high concentration to the cell suspension containing the cell mass detached from the substrate.

Each treatment is described in detail below.

(Cell Singulation Treatment)

Herein, the “singulated cells” mean cells each existing as one cell (also called a single cell) without aggregation between a plurality of cells. The “cell singulation treatment” refers to treatment for dissociating a cell group made up of a plurality of cells, such as a cell mass or a cell associated body, into individual cells.

(Cell Singulation Treatment Involving Passing Cell Suspension Through Mesh Filter)

An example of the cell singulation treatment in the step 3 is performed by passing a cell suspension through a mesh filter.

For example, the cell singulation treatment is performed by passing the cell suspension containing the cell mass detached from the substrate through a mesh filter after the cells have been mildly detached from the substrate by using the cell detachment solution containing a metal ion chelating agent and using the ultrasonic vibration. Thus, the cell mass can be dissociated into individual cells to prepare single cells while maintaining a high survival rate.

The mesh filter to be used in this case is not particularly limited as long as the mesh filter can be sterilized, and examples thereof include a nylon mesh and a metal-made mesh formed of a metal such as stainless steel. The aperture of the mesh only needs to be such a size that the cell mass can be turned into single cells, and a filter size may be appropriately selected in accordance with the size of the cells and intercellular binding properties, though varying depending on the kind of the cells.

The shape of the mesh in the mesh filter, that is, the shape of its perforation through which cells are passed is not particularly limited as long as the shape allows the passage of single cells. Accordingly, the definition of the mesh size of the mesh filter differs in accordance with the shape of the mesh. For example, when the shape of the mesh is a square, the mesh size may be defined by the length of a side or a diagonal line. When the shape of the mesh is a rectangle, the mesh size may be defined by a long side, a short side, a diagonal line, or their average length. When the shape of the mesh is an ellipse, the mesh size may be defined by the major axis, the minor axis, or their average length. When the shape of the mesh is a circle, the mesh size may be defined by the aperture (diameter). The mesh size may be measured by observation with a microscope.

For example, the aperture of the nylon mesh may be 10 μm or more and 100 μm or less, preferably 20 μm or more and 70 μm or less, particularly preferably 20 μm or more and 40 μm or less. Particularly when a mesh filter having an aperture of 20 μm or more and 40 μm or less is used, an appropriate shear force can be applied to the cell mass, and hence, as shown in Examples to be described later, the singulation of the cells and a high survival rate thereof can both be achieved.

As a method of passing the cell suspension through the mesh filter, there is given a method involving recovering the cell suspension from the culture dish and passing the cell suspension through the mesh filter by using a dispenser. When the cell suspension containing a metal ion chelating agent, or containing a metal ion chelating agent and serum is passed through the mesh filter, the cell mass is dissociated upon passage through the mesh filter, and thus cell singulation is achieved. In the resultant cell suspension, the cells have been sufficiently singulated, and can be brought into a state suitable in the subsequent process of cell counting. In addition, when the cell suspension containing the singulated cells thus obtained is seeded in an appropriate culture substrate again, the obtained cells can be effectively maintained and grown.

The number of times the cell suspension is passed through the mesh filter is not particularly limited as long as the number is at least 1 or more, but the passage may be repeated to the extent that the cells are not damaged. In addition, the passage may be performed with a combination of sizes of mesh filters. For example, it is also effective that the cell suspension is first passed through a 40 μm mesh filter and then passed through a 20 μm mesh filter.

(Method Involving Passing Cell Suspension Containing Serum Through Mesh Filter)

An example of the cell singulation treatment in the step 3 is performed by passing a cell suspension containing serum through a mesh filter.

After the cells have been mildly detached from the substrate by using the cell detachment solution containing a metal ion chelating agent and using the ultrasonic vibration, serum is added to the cell detachment solution. When the thus obtained cell suspension containing the serum together with the cell mass is passed through the mesh filter, the cell mass can be dissociated to prepare singulated cells while maintaining a high survival rate. The final concentration of the serum in the cell suspension may be set to 0.1% or more and 90% or less, and is preferably 1% or more and 20% or less from the viewpoints of the handling of the solution and the cost of the serum. For example, a general cell culture medium often contains 10% of serum, and when, at the time of the addition of serum to the above-mentioned cell detachment solution, an equal amount of the cell culture medium containing serum is added to the cell detachment solution, the final concentration of the serum in the cell suspension becomes 5%. Serum having an appropriate concentration can contribute to improving the survival rate of the cells.

The serum to be used in this case is not particularly limited as long as the serum contains a protein derived from serum that can be used in cell culture, and examples thereof include serum itself and a main component thereof such as albumin. Examples of the serum include FBS, horse serum, goat serum, and human serum.

FBS is conveniently used because of its ease of availability. As shown in Examples to be described later, the use of serum is effective for achieving the singulation of the cells and a high survival rate thereof.

(Cell Singulation Treatment Performed Using Metal Ion Chelating Agent at Concentration of 1.0 mM or More)

An example of the cell singulation treatment in the step 3 is performed by adjusting the concentration of the metal ion chelating agent in a cell suspension containing the cells detached in the above-mentioned step 2 to 1.0 mM or more, and then applying a shear force to the cells.

After the cells have been mildly detached from the substrate by using the cell detachment solution containing a metal ion chelating agent and using the ultrasonic vibration, a metal ion chelating agent is added to the cell suspension containing the cells detached from the substrate so as to have a concentration of 1.0 mM or more.

The metal ion chelating agent to be added to the cell suspension after the step 2 is not limited as long as the metal ion chelating agent has an action of dissociating intercellular binding, but an example thereof is ethylenediaminetetraacetic acid. When ethylenediaminetetraacetic acid is used as the chelating agent, the pH of the cell suspension is preferably 7.0 or more and 8.0 or less. This is because, when the pH is higher within the neutral region, in which the survival rate can be kept high, the chelating ability of ethylenediaminetetraacetic acid can be enhanced, and hence the dissociation of the cells can be further promoted.

The content of the chelating agent in the cell suspension is preferably 1.0 mM or more and 5.0 mM or less. When the content falls within this range, a chelating effect can be reliably obtained, and besides, a reduction in activity due to the presence of an excess of the chelating agent can be suppressed.

In addition, this cell singulation treatment includes an operation of generating a convective flow or the like in the cell detachment solution to apply a shear force to the cells in addition to an operation of adjusting the concentration of the metal ion chelating agent in the cell detachment solution to 1.0 mM or more.

As a method of generating a convective flow in the cell detachment solution, there are given, for example, pipetting and use of a pump or a stirring blade. The shear force on the cells is applied by, for example, passing the cell suspension through a small tube. For example, a pipette tip may be used as the small tube through which the cell suspension is to be passed. Through the operation of applying a shear force to the cells, the dissociation of the cell mass into single cells can be promoted, and the influence of the metal ion chelating agent on the cells can be reduced. In order to further promote the singulation of the cells from the cell mass, the cell suspension may be further passed through a mesh filter after the cell singulation treatment using the metal ion chelating agent at a concentration of 1.0 mM or more. The inner diameter of the small tube, or the inner diameter (smallest portion) of the pipette tip is preferably 0.1 mm or more and 5 mm or less, more preferably 0.15 mm or more and 2 mm or less from the viewpoints of an appropriate shear force on the cells, the maintenance of the survival rate, and the water permeability of the cell suspension.

(Flow of Method of Recovering Cells According to Embodiment of the Present Invention)

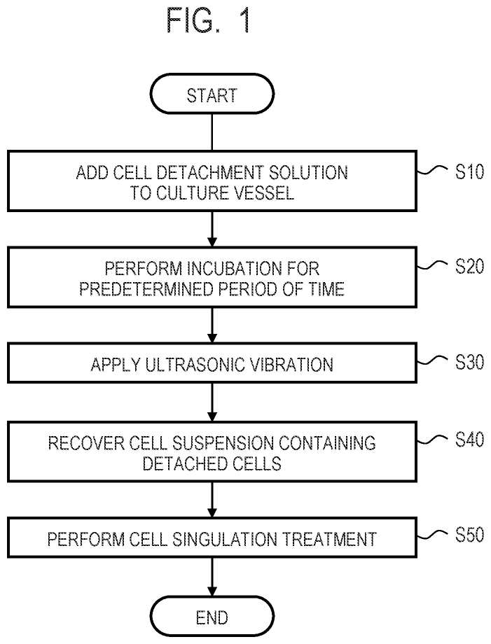

An example of the flow of the method of recovering cells according to the embodiment of the present invention is illustrated in FIG. 1. For example, the above-mentioned step 1 includes Step S10 of adding a cell detachment solution to a culture vessel, and Step S20 of performing incubation for a predetermined period of time while maintaining a state in which cells are brought into contact with the cell detachment solution. In addition, for example, the above-mentioned step 2 includes Step S30 of applying ultrasonic vibration in order to detach the cells from the substrate, and may further include Step S40 of recovering a cell suspension containing the cells detached from the substrate. In addition, for example, the above-mentioned step 3 includes Step S50 of performing cell singulation treatment. The flow of the method of recovering cells according to the embodiment of the present invention may further include a step of observing the state of the cells. Herein, “performing incubation” refers to performing a step including at least any one of a step of adjusting a temperature, a step of adjusting a humidity, and a step of adjusting air composition. The “step of adjusting a temperature” is, for example, a step of controlling a heater so that the temperature of the surroundings of the cell culture vessel may be around 37° C. The “step of adjusting a humidity” is, for example, a step of adjusting the humidity to 90% or more by placing a humidifying vat so as to prevent the medium from being dried. The “step of adjusting air composition” is, for example, a step of supplying CO2 so as to achieve a CO2 partial pressure of around 5% in order to prevent the medium from being oxidized.

(Configuration of Cell Recovery Apparatus)

A cell recovery apparatus according to an embodiment of the present invention is a cell recovery apparatus configured to recover cells cultured on a substrate, and includes a medium exchange section, an ultrasonic irradiation section, and a singulated cell obtaining section. The exchange section is configured to add a cell detachment solution containing a metal ion chelating agent to the substrate having the cells adhering thereto to bring the cells into contact with the cell detachment solution. In addition, the ultrasonic irradiation section is configured to apply ultrasonic vibration to the substrate and the cells. In addition, the singulated cell obtaining section is configured to obtain the cells singulated from the cells detached from the substrate.

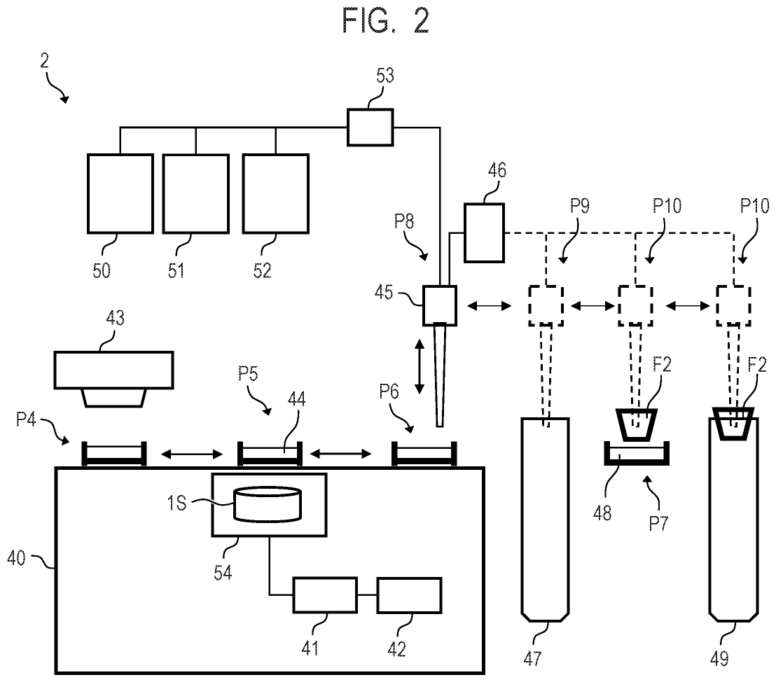

The configuration of a cell recovery apparatus 2 according to the embodiment of the present invention is described with reference to a conceptual view illustrated in FIG. 2.

A culture substrate movement control section 40 is a unit configured to hold and move a culture substrate 44.

The culture substrate movement control section 40 may include a warmer configured to control the temperature and humidity of an environment in which detachment of cells from the substrate is performed, and examples of such warmer include a heating unit capable of keeping a temperature of 37° C. and a humidifying unit. For example, an XYZ motorized stage is used to move the culture substrate 44. The culture substrate movement control section 40 horizontally moves the culture substrate 44 among a position P4, a position P5, and a position P6. At the position P4, an image of cells cultured in the culture substrate 44 can be acquired. At the position P5, the inside of the culture substrate 44 is irradiated with an ultrasonic wave by an ultrasonic irradiation section 1S. In addition, at the position P6, by a pipetting section 45, a solution in the culture substrate 44 is recovered, or any of various solutions are added to the culture substrate 44.

Further description is made taking an example of a process of cell detachment using an ultrasonic wave.

Cells are cultured in the culture substrate 44, and when a timing at which the cells are to be detached is reached, the culture substrate 44 is arranged at the position P6. As appropriate, through use of the culture substrate movement control section 40, the culture substrate 44 may be arranged at the position P4 for the observation of the state of the cells by a cell observation section 43. The culture substrate 44 arranged at the position P6 has the cells adhering thereto.

First, the medium needs to be removed and exchanged with the cell detachment solution. Accordingly, at the position P6, the medium in the culture substrate 44 is recovered by the pipetting section 45. The recovered medium is discarded into a waste liquid recovery section 47 at a position P9. Various media are stocked in a medium 50 for washing, a medium 51 for culture, and a cell detachment solution 52, and are transported to the pipetting section 45 by a liquid transportation control section 53. The pipetting section 45 can gain access to the culture substrate 44 by moving in a vertical direction. As appropriate, the cell recovery apparatus 2 may include a lid opening and closing system for the culture substrate 44.

After the medium has been removed, the cells adhering to the culture substrate 44 may be washed as appropriate.

The medium 50 for washing is transported to the pipetting section 45 by the liquid transportation control section 53, and at the position P6, the medium 50 for washing is added into the culture substrate 44 by the pipetting section 45. The medium 50 for washing is removed in the same manner as in the above-mentioned medium removal method, and then the cell detachment solution 52 is transported to the pipetting section 45 by the liquid transportation control section 53. Subsequently, at the position P6, the cell detachment solution 52 is added into the culture substrate 44 by the pipetting section 45.

Next, the culture substrate movement control section 40 moves the culture substrate 44 to the position P5. Here, the culture substrate 44 is set in the ultrasonic irradiation section 1S. A hitherto known configuration may be used as the ultrasonic irradiation section 1S. A configuration including the ultrasonic irradiation section 1S is exemplified by a form combining an ultrasonic detachment unit 54 including the ultrasonic irradiation section 1S with a function generator 42 and an amplifier 41 which are configured to input, for example, an arbitrary frequency and voltage into the ultrasonic detachment unit 54.

The culture substrate 44 is irradiated with an ultrasonic wave from the ultrasonic irradiation section 1S to detach the cells adhering to the culture substrate 44. The cells detached from the culture substrate 44 include cell masses, and hence cell singulation treatment is then performed.

First, the culture substrate movement control section 40 moves the culture substrate 44 to the position P6. At the position P6, the cell suspension in the culture substrate 44, which contains the cells detached from the culture substrate 44, is recovered by the pipetting section 45. The recovered cell suspension is passed through a mesh filter F2 at a position P10 and seeded in a detached cell recovery section 48, for example, a fresh cell culture substrate placed at a position P7. Alternatively, at another position P10, the cell suspension is passed through the mesh filter F2 and recovered in a tube 49 serving as the detached cell recovery section 48. The recovered cells have been singulated, and are used for later cell culture or assay such as cell counting.

The cell recovery apparatus 2 may include the cell observation section 43 configured to acquire an image of the cells cultured in the culture substrate 44, allowing a cell detachment state and a cell recover state to be recognized.

The cell recovery apparatus 2 may include a control section configured to control the above-mentioned cell detachment process. The control section is a unit configured to control the operation of each section in the cell recovery apparatus 2. For example, the control section may be formed of a computer including an arithmetic processing section such as a CPU, a memory such as a RAM, and a storage section such as a hard disk drive. The control section may include an operation section.

The operation section may include, for example, a display section and an input section. The display section can display image data output from the control section, and various kinds of information concerning the operation of the cell recovery apparatus 2. For example, a liquid crystal display is used for the display section. The input section accepts various kinds of instruction input from a user. For example, a keyboard and a mouse are used for the input section. Both of the function of the display section and the function of the input section may be in a single unit such as a touch panel display.

### EXAMPLES

The present invention is described below in more detail by way of Examples and Comparative Examples. However, the present invention is by no means limited to the following Examples and Comparative Examples.

<Preparation Example of Cell Detachment Solution 1>

A hydrophilic polymer and ethylenediaminetetraacetic acid were added to divalent cation-free phosphate buffered saline (PBS(−), manufactured by Thermo Fisher Scientific) at concentrations of 1.0 mass % and 0.5 mmol/L, respectively, and were completely dissolved therein. PEG2000 having a peak molecular weight Mp measured by GPC of 2,030 (manufactured by Kishida Chemical Co., Ltd.) was used as the hydrophilic polymer. Further, the pH of the solution was adjusted to 7.4 using hydrochloric acid and sodium hydroxide, and the resultant was used as a cell detachment solution 1. The viscosity of the cell detachment solution 1 was 1.22 mPas.

<Preparation Example of Cell Detachment Solution 2>

A hydrophilic polymer and ethylenediaminetetraacetic acid were added to divalent cation-free phosphate buffered saline (PBS(−), manufactured by Thermo Fisher Scientific) at concentrations of 1.0 mass % and 0.1 mmol/L, respectively, and were completely dissolved therein. PEG4000 having a peak molecular weight Mp measured by GPC of 3,700 (manufactured by Kishida Chemical Co., Ltd.) was used as the hydrophilic polymer. Further, the pH of the solution was adjusted to 7.4 using hydrochloric acid and sodium hydroxide, and the resultant was used as a cell detachment solution 2. The viscosity of the cell detachment solution 2 was 1.23 mPa·s.

### Example 1

### (Culture of Cells on Substrate)

Human mesenchymal stem cells (hMSCs) were seeded in a Φ60 polystyrene dish (manufactured by Corning Incorporated) at a density of 4,000 cells/cm², and were cultured under an environment at 37° C. and a CO2 concentration of 5%. MSCGM Bullet Kit (manufactured by Lonza) was used as a medium. After 96 hours of culture, the medium was exchanged, and the cells were then cultured up to 168 hours. The state of the cells was observed with a phase contrast microscope to recognize cell adhesion and growth. The cell coverage area ratio of the dish was about 80%.

(Detachment of Cells from Substrate)

The medium in the dish was removed, and the cells were washed with PBS(−) and then immersed in the above-mentioned cell detachment solution 1 for 10 minutes. After that, the dish was set in an ultrasonic detachment apparatus, and sweep vibration (frequency: 22-27 kHz, sweep period: 1 s, voltage: 100 V) was applied at an environmental temperature of 37° C. for 3 minutes to detach the cells from the substrate.

(Cell Singulation Treatment)

The cell suspension in such a state that cell masses were suspended in the cell detachment solution 1 was aspirated with a pipetter, and the cell suspension was passed through a mesh filter (mesh size: 20 μm) that had been set on a 15 mL tube in advance while the tip of the pipette was lightly pressed against the mesh filter. After that, the cell suspension that had passed through the mesh filter was recovered.

The resultant cell suspension was placed in a hemocytometer and observed in four fields of view (1 mm² per field of view) with a microscope, and as a result, no cell mass was found. The number of cells was measured using the hemocytometer, and the survival rate of the cells was calculated using a viability determination method based on trypan blue staining. As a result, the survival rate of the cells was found to be 80%. Thus, cell singulation treatment using a mesh filter was able to be performed.

### Comparative Example 1

Cells were detached from a dish by applying ultrasonic vibration in the same manner as in Example 1. The cell suspension in such a state that cell masses were suspended in the cell detachment solution 1 was placed in a hemocytometer and observed in four fields of view with a microscope, and as a result, a large number of cell masses were found. In addition, the survival rate of the cells was calculated in the same manner as in Example 1, and as a result, the survival rate of the cells was found to be 79%. When cell singulation treatment was not performed, singulated cells were not obtained. Accordingly, it was difficult to count the number of cells.

### Example 2

Cells were detached by applying ultrasonic vibration in the same manner as in Example 1. Subsequently, before cell singulation treatment was performed, serum (solution containing 10% of serum, that is, MSCGM Bullet Kit) was added to the cell suspension in such a state that cell masses were suspended in the cell detachment solution 1. Here, the final concentration of the serum in the cell suspension became 5%. Next, the cell suspension was aspirated with a pipetter, and the cell suspension was passed through a mesh filter (mesh size: 20 μm) that had been set on a 15 mL tube in advance while the tip of the pipette was lightly pressed against the mesh filter. After that, the cell suspension that had passed through the mesh filter was recovered.

The resultant cell suspension was placed in a hemocytometer and observed in four fields of view with a microscope, and as a result, no cell mass was found. The number of cells was measured using the hemocytometer, and the survival rate of the cells was calculated using a viability determination method based on trypan blue staining. As a result, the survival rate of the cells was found to be 90%. Thus, the cells were able to be singulated with the mesh filter. In the cell singulation treatment, the addition of the serum was able to improve the survival rate of the cells.

### Example 3

Cell detachment and cell singulation treatment were performed in the same manner as in Example 2 except that, in Example 2, the mesh size of the mesh filter was changed to 10 μm. The resultant cell suspension was placed in a hemocytometer and observed in four fields of view with a microscope, and as a result, no cell mass was found. In addition, the survival rate of the cells was calculated in the same manner as in Example 2, and as a result, the survival rate of the cells was found to be 83%. Thus, the cells were able to be singulated with the mesh filter.

### Example 4

Cell detachment and cell singulation treatment were performed in the same manner as in Example 2 except that, in Example 2, the mesh size of the mesh filter was changed to 30 μm. The resultant cell suspension was placed in a hemocytometer and observed in four fields of view with a microscope, and as a result, no cell mass was found. In addition, the survival rate of the cells was calculated in the same manner as in Example 2, and as a result, the survival rate of the cells was found to be 93%. Thus, the cells were able to be singulated with the mesh filter.

### Example 5

Cell detachment and cell singulation treatment were performed in the same manner as in Example 2 except that, in Example 2, the mesh size of the mesh filter was changed to 40 μm. The resultant cell suspension was placed in a hemocytometer and observed in four fields of view with a microscope, and as a result, no cell mass was found. In addition, the survival rate of the cells was calculated in the same manner as in Example 2, and as a result, the survival rate of the cells was found to be 94%. Thus, the cells were able to be singulated with the mesh filter.

### Example 6

Cell detachment and cell singulation treatment were performed in the same manner as in Example 2 except that, in Example 2, the mesh size of the mesh filter was changed to 50 μm. The resultant cell suspension was placed in a hemocytometer and observed in four fields of view with a microscope. As a result, singulated cells were partially found, and a cell mass having a diameter of 50 μm or more was also found. In addition, the survival rate of the cells was calculated in the same manner as in Example 2, and as a result, the survival rate of the cells was found to be 92%.

### Example 7

Cell detachment and cell singulation treatment were performed in the same manner as in Example 2 except that, in Example 2, the mesh size of the mesh filter was changed to 70 μm. The resultant cell suspension was placed in a hemocytometer and observed in four fields of view with a microscope. As a result, singulated cells were partially found, and a cell mass having a diameter of 50 μm or more was also found. In addition, the survival rate of the cells was calculated in the same manner as in Example 2, and as a result, the survival rate of the cells was found to be 90%.

### Comparative Example 2

Cells were detached from a dish by applying ultrasonic vibration in the same manner as in Example 2. After that, serum was added to the cell suspension in such a state that cell masses were suspended in the cell detachment solution 1, and the resultant was recovered. After that, the operation of passing the cell suspension through a mesh filter was not performed, and the cell suspension was placed in a hemocytometer and observed in four fields of view with a microscope, and as a result, a large number of cell masses were found. Thus, the cells were not able to be singulated. In addition, the survival rate of the cells was calculated in the same manner as in Example 2, and as a result, the survival rate of the cells was found to be 92%.

### Example 8

The cell suspensions obtained after the cell singulation treatments in Examples 2 to 4 and 6, and after the detachment of the cells from the dish in Comparative Example 2 were each seeded in a fresh substrate (Corning dish). At this time, the number of cells to be seeded was set to 10,000. After that, the cells were cultured under an environment at 37° C. and a CO2 concentration of 5% for 5 days. After the culture, the cells were detached from the substrate through use of trypsin, and the number of cultured cells was measured.

The results are shown in Table 1.

A mesh size in Table 1 represents the mesh size of the mesh filter used in the cell singulation treatment in each Example. In addition, the number of cells after culture in Table 1 represents the number of cells after 5 days of culture.

TABLE 1

| Origin of | Mesh | Number of |
|---|---|---|
| cell suspension | size (μm) | cells after culture |
| Example 2 | 20 | 94,600 |
| Example 3 | 10 | 76,800 |
| Example 4 | 30 | 84,900 |
| Example 6 | 50 | 70,200 |
| Comparative Example 2 | — | 76,200 |

As apparent from Table 1, the cells obtained through the cell singulation treatment with the mesh filter in each of Examples 2 to 4 and 6 were not only singulated, but also showed no problem with growth in the subsequent culture. In Example 2, in which a mesh filter having a mesh size of 20 μm was used, and Example 4, in which a mesh filter having a mesh size of 30 μm was used, higher numbers of cells were obtained than in Comparative Example 2, in which cell singulation treatment with a mesh filter was not performed.

### Example 9

Cells were detached from a dish by applying ultrasonic vibration in the same manner as in Example 1. Before cell singulation treatment was performed, ethylenediaminetetraacetic acid for cell singulation treatment was added to the cell suspension in such a state that cell masses were suspended in the cell detachment solution 1. The final concentration of ethylenediaminetetraacetic acid in the cell suspension was set to 5 mM. After the addition of ethylenediaminetetraacetic acid to the cell suspension, the mixture was left to stand at 37° C. for 15 minutes. Next, the cell suspension was stirred by pipetting.

The resultant cell suspension was placed in a hemocytometer and observed in four fields of view with a microscope, and as a result, no cell mass was found. In addition, the survival rate of the cells was calculated in the same manner as in Example 1, and as a result, the survival rate of the cells was found to be 92%. Thus, the cells were able to be singulated with ethylenediaminetetraacetic acid.

### Comparative Example 3

Cell detachment and cell singulation treatment (only a pipetting operation) were performed in the same manner as in Example 9 except that, in Example 9, ethylenediaminetetraacetic acid for cell singulation treatment was not added to the cell suspension. The detached cells were cell masses, and singulated cells were not obtained.

### Comparative Example 4

Cell detachment was performed in the same manner as in Example 1 except that, in Example 1, PBS(−) was used in place of the cell detachment solution 1. As a result, the cells were hardly detached from the substrate.

In addition, part of the detached cells were cell masses, and singulated cells were not obtained.

It is found from the foregoing that, in the method of recovering cells according to Examples of the present invention, the cell detachment solution to be used in the step 1 needs to contain a metal ion chelating agent.

### Comparative Example 5

Cell detachment was performed in the same manner as in Example 1 except that, in Example 1, the ultrasonic vibration was not applied. However, the cells were hardly detached from the substrate. In addition, part of the detached cells were cell masses, and singulated cells were not obtained.

It is found from the foregoing that, in the method of recovering cells according to Examples of the present invention, the ultrasonic vibration to be used in the step 2 is needed.

### Example 10

Mouse skeletal muscle myoblasts (hereinafter abbreviated as “C2C12 cells”) were seeded in a Φ35 polystyrene dish (manufactured by Corning Incorporated) at a density of 10,000 cells/cm², and were cultured under an environment at 37° C. and a CO2 concentration of 5%. DMEM (manufactured by Thermo Fisher Scientific) supplemented with the following two materials was used as a medium.
Fetal Bovine Serum (manufactured by Sigma-Aldrich), final concentration: 10 mass %
Penicillin-streptomycin (10,000 U/ml, manufactured by Thermo Fisher Scientific), final concentration: 1 mass %

The culture was performed for 48 hours, and the state of the cells was observed with a phase contrast microscope to recognize cell adhesion and growth. The cell coverage area ratio of the dish was about 80%.

Next, cell detachment and cell singulation treatment were performed in the same manner as in Example 2 except that, in Example 2, the cells were changed to the C2C12 cells, the cell detachment solution was changed to the cell detachment solution 2, and the solution containing serum was changed to DMEM containing 10% of serum. The resultant cell suspension was placed in a hemocytometer and observed in four fields of view with a microscope, and as a result, no cell mass was found. In addition, the survival rate of the cells was calculated in the same manner as in Example 1, and as a result, the survival rate of the cells was found to be 85%. Thus, the cells were able to be singulated with the mesh filter. Further, also when mesh filters having mesh sizes of 40 μm and 70 μm were used, similar survival rates (86% and 87%, respectively) were obtained, and no cell masses were found.

According to the present invention, the method of recovering cells and the cell recovery apparatus each of which is capable of turning a cell mass detached from a substrate into singulated cells effectively and with a high survival rate in a method involving detaching cells from the substrate through use of an ultrasonic wave and recovering the singulated cells can be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application Nos. 2022-040723, filed Mar. 15, 2022, and 2022-197317, filed Dec. 9, 2022, which are hereby incorporated by reference herein in their entirety.

What is claimed is:

1. A method of recovering cells cultured on a substrate, the method comprising:
a step 1 of adding a cell detachment solution containing a metal ion chelating agent to the substrate having the cells adhering thereto to bring the cells into contact with the cell detachment solution;
a step 2 of applying ultrasonic vibration to the substrate and the cells to detach the cells from the substrate; and
a step 3 of obtaining the cells singulated from the cells detached in the step 2.

2. The method of recovering cells according to claim 1, wherein the step 3 comprises passing a cell suspension containing the cells detached in the step 2 through a mesh filter.

3. The method of recovering cells according to claim 2, wherein the mesh filter has a mesh size of 20 μm or more and 40 μm or less.

4. The method of recovering cells according to claim 1, wherein the step 3 comprises adjusting a concentration of the metal ion chelating agent in a cell suspension containing the cells detached in the step 2 to 1.0 mM or more, and then applying a shear force to the cells.

5. The method of recovering cells according to claim 4, wherein the shear force is applied by passing the cell suspension through a small tube.

6. The method of recovering cells according to claim 5, wherein the small tube is a pipette tip.

7. The method of recovering cells according to claim 1, further comprising, before the step 3, adding serum to a cell suspension containing the cells detached in the step 2.

8. The method of recovering cells according to claim 1, wherein the cell detachment solution contains the metal ion chelating agent at 0.01 mM or more and 5.0 mM or less.

9. The method of recovering cells according to claim 1, wherein the cell detachment solution is free of any protease.

10. The method of recovering cells according to claim 1, wherein the cells are stem cells.

11. The method of recovering cells according to claim 1, wherein the step 2 is performed under a condition of an environmental temperature of 30.0° C. or more and 37.5° C. or less.

12. A cell recovery apparatus configured to recover cells cultured on a substrate, the cell recovery apparatus comprising:
a medium exchange section configured to add a cell detachment solution containing a metal ion chelating agent to the substrate having the cells adhering thereto to bring the cells into contact with the cell detachment solution;
an ultrasonic irradiation section configured to apply ultrasonic vibration to the substrate and the cells; and
a singulated cell obtaining section configured to obtain the cells singulated from the cells detached from the substrate.

13. The cell recovery apparatus according to claim 12, wherein the singulated cell obtaining section includes a mesh filter.
